(19) United States
(12) Patent Application Publication
(10) Pub. No.: US 2026/0232490 A1
(43) Pub. Date: Aug. 13, 2026

(54) ROTATIONAL MEMBRANE CUTTER

(71) Applicant: Alcon Inc., Fribourg (CH)

(72) Inventors: Reto Grüebler, Greifensee (CH); Christoph Siegenthaler, Eschenz (CH)

(21) Appl. No.: 19/457,478

(22) Filed: Jan. 23, 2026

Related U.S. Application Data
(60) Provisional application No. 63/756,390, filed on Feb. 10, 2025.

(51) Int. Cl. A61F 9/007

(52) U.S. Cl. CPC A61F 9/00763

(57) ABSTRACT

An apparatus for eye surgery, which may comprise a tube, a base having a plurality of base teeth coupled to the base, and a blade. The blade can be configured to move around an axis of rotation relative to the base teeth parallel to a shearing plane. The shearing plane can be orthogonal to the axis of rotation, and oriented at an obtuse angle relative to a longitudinal axis of the tube. A driver can be configured to oscillate the blade parallel to the shearing plane. The base may comprise a surface configured to interface with a retina and may have a curved profile. The base profile may have a center of curvature that is coincident with the axis of rotation. Some embodiments of the base profile may be characterized as an arc, circular arc, or semicircle.

### TECHNICAL FIELD

The invention set forth in the appended claims relates generally to ophthalmic surgery, including, without limitation, apparatuses, systems, and methods for delaminating retinal membrane.

### BACKGROUND

The human eye can suffer a variety of maladies, which can cause mild deterioration to complete loss of vision. While contact lenses and eyeglasses can compensate for some ailments, ophthalmic surgery may be required for others.

For example, various macular surface diseases can cause a membrane to form on the surface of a retina. Different types of membranes include internal limiting membranes (ILM), epi-retinal membranes (ERM), and proliferative membranes. Each of these membranes may develop because of a different eye disease or condition. In the case of ERMs, for example, the scar tissue formation can be associated with several ocular conditions, such as prior retinal tears or detachments, or retinal vascular diseases, such as diabetic retinopathy or venous occlusive disease. ERMs can also be developed due to trauma associated with ocular surgery or be associated with intraocular inflammation. In another example, proliferative membranes may be caused by diabetic retinopathy, which in its advanced form can cause new abnormal blood vessels to proliferate on the surface of the retina. Some membranes can induce traction on the retina, which can lead to further complications.

Surgical removal of the membrane can be a useful treatment for such conditions. While the benefits of surgical removal are known, improvements to surgical systems, components, and processes can continue to improve outcomes and benefit patients.

### BRIEF SUMMARY

New and useful systems, apparatuses, and methods for removing retinal membranes are set forth in the appended claims. Illustrative embodiments are also provided to enable a person skilled in the art to make and use the claimed subject matter.

For example, some embodiments can be used to sever connective tissue between a retina and a membrane. In more particular embodiments, the connective tissue may be sheared in a plane perpendicular to movement of the apparatus. Some embodiments may comprise a base portion and a blade portion. The base portion can remain fixed relative to the blade portion, which can protect the retina from movement of the blade portion.

One or both parts can contain teeth in some embodiments. The teeth can vary in number and form. For example, some teeth may comprise or consist essentially of a triangular, rounded, or circular geometry. In some embodiments, the base may have teeth that are rounded to protect the retina, and the blade may have teeth that are pointed.

Small teeth with high cut rates can minimize inducing traction during membrane delamination. Connective tissue may be severed by moving the blade teeth relative to the base teeth. For example, the blade portion can be connected to an oscillating shaft, while the base can be coupled to an outer tube. In some embodiments, the base and the blade may be used as a spatula.

Additionally, or alternatively, some embodiments may have an oscillating shaft that can be configured for aspirating tissue. For example, the shaft may have a hole above the blade teeth.

More generally, some embodiments may comprise an apparatus for eye surgery, which may comprise a tube having an opening, a base coupled to the tube adjacent to the opening, a plurality of base teeth coupled to the base, and a blade disposed at least partially within the opening between the tube and the base, wherein the blade is configured to move around an axis of rotation relative to the base teeth. The base may comprise a surface configured to interface with a retina. Each of the base teeth may comprise a leading edge, and the leading edges can define a base profile that is curved. In more particular embodiments, the base profile may have a center of curvature that is coincident with the axis of rotation. For example, some embodiments of the base profile may be characterized as an arc, circular arc, or semicircle having a center of curvature that is coincident with the axis of rotation.

In some embodiments, the apparatus may further comprise a shaft coupled to the blade, and the shaft may be rotatably disposed at least partially within the tube. Additionally, or alternatively, some embodiments of the blade may comprise a plurality of blade teeth, which may be configured to oscillate relative to the base teeth. Each of the blade teeth may comprise a leading edge, and the leading edges of the blade teeth may define a blade profile. The blade profile may be a curve, which may be similar to the base profile. For example, the base profile may be a first arc and the blade profile may be a second arc. In some embodiments, the first arc and the second arc may be congruent. Additionally, or alternatively, the leading edges of the base teeth, the leading edges of the blade teeth, or both may have a thickness based the surgical need, condition of the membrane / retina, etc. For example, in some embodiments, the thickness may be approximately in a range of 30 to 400 micrometers. In some embodiments, the thickness may be less than 400 micrometers. As a specific example, the thickness may be approximately 200 micrometers. In other embodiments, the thickness may be approximately 100 micrometers. Other dimensions are also contemplated (e.g., the thickness may be in a range of approximately 30 to 100 micrometers, approximately in a range of 100 to 300 micrometers, approximately in a range of 300 to 400 micrometers, approximately in a range of 400 to 800 micrometers, approximately in a range of 800 to 1600 micrometers, etc.)

In some embodiments, the tube has a longitudinal axis, and the base teeth and the blade teeth define a shearing plane, which can be oriented relative to the longitudinal axis at an obtuse angle.

In some embodiments, the blade teeth may be oscillated through manual or hand actuated motion (e.g., the user squeezing, pressing, or in some way actuating an actuator on the device to move the blade teeth). In yet other embodiments, a driver can be configured to be coupled to the shaft, and the driver can rotate the shaft to oscillate the blade teeth parallel to the shearing plane. The frequency of shaft rotation may be slow (e.g., in a range of 1 to 500 Hertz (Hz)) or may be fast (e.g., in a range of 1000 to 20,000 Hz or more) depending on the surgical need, condition of the membrane / retina, etc. Other frequencies are also contemplated (e.g., between 500 and 1000 Hz). In some embodiments, the frequency may be fixed or may be adjusted by the user.

Features, elements, and aspects described in the context of some embodiments may also be omitted, combined, or replaced by alternative features. Other features, objectives, advantages, and a preferred mode of making and using the claimed subject matter are described in greater detail below with reference to the accompanying drawings of illustrative embodiments.

### BRIEF DESCRIPTION OF THE DRAWINGS

The accompanying drawings illustrate some objectives, advantages, and a preferred mode of making and using some embodiments of the claimed subject matter. Like reference numbers represent like parts in the examples.

FIG. 1 is an isometric view of an apparatus for cutting membrane during ophthalmic surgery.

FIG. 2 is an assembly view of the apparatus of FIG. 1.

FIG. 3 is a detail view of the apparatus of FIG. 1.

FIG. 4 is an assembly view of the head of the apparatus of FIG. 3.

FIG. 5 is an isometric view of the head of FIG. 4 in an assembled configuration.

FIG. 6 is a bottom view of the head of FIG. 5.

FIG. 7 is a partial side view of the apparatus of FIG. 1.

FIG. 8 is an isometric view of another example of the apparatus.

FIG. 9 is an assembly view of the apparatus of FIG. 8.

FIG. 10 is a detail view of the apparatus of FIG. 8.

FIG. 11 is an assembly view of a portion of the apparatus of FIG. 8.

FIG. 12 is an isometric view of a portion of the apparatus including the head of FIG. 8 in an assembled configuration.

FIG. 13 is a bottom view of the head of FIG. 12.

FIG. 14 is a partial side view of the apparatus of FIG. 8.

### DESCRIPTION OF EXAMPLE EMBODIMENTS

The following description of example embodiments provides information that enables a person skilled in the art to make and use the subject matter set forth in the appended claims, but it may omit certain details already well known in the art. The following detailed description is, therefore, to be taken as illustrative and not limiting.

The example embodiments may also be described herein with reference to spatial relationships between various elements or to the spatial orientation of various elements depicted in the attached drawings. In general, such relationships or orientation assume a frame of reference consistent with or relative to a patient in a position for ophthalmic surgery. However, as should be recognized by those skilled in the art, this frame of reference is merely a descriptive expedient rather than a strict prescription.

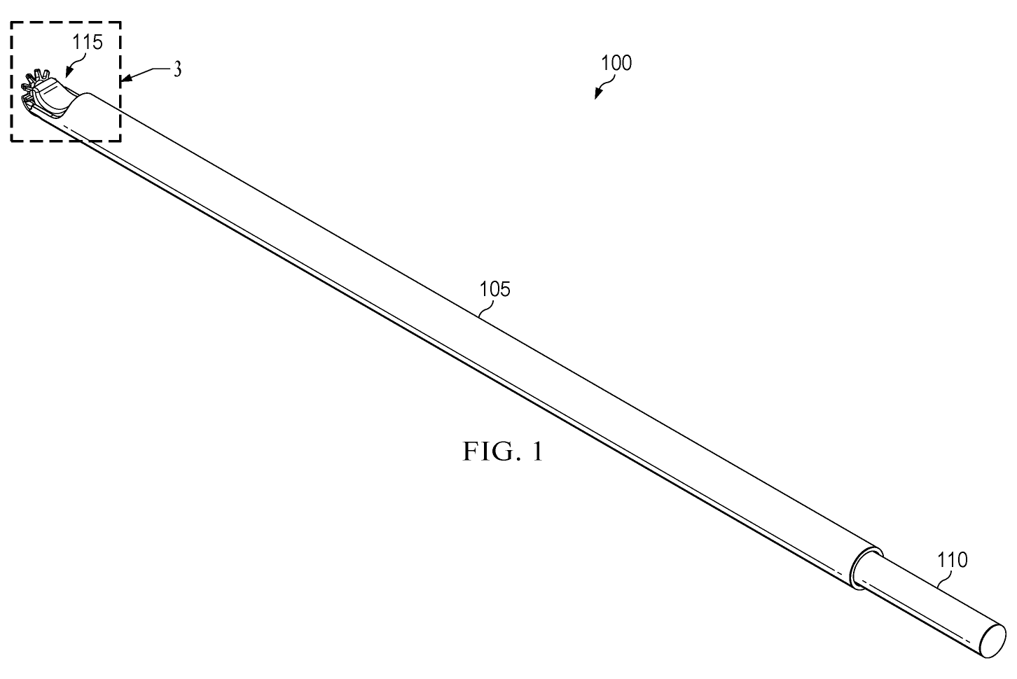

FIG. 1 is an isometric view of an apparatus 100 for cutting membrane during ophthalmic surgery. The apparatus 100 of FIG. 1 generally comprises a tube 105, a shaft 110 rotatably disposed at least partially within the tube 105, and a head 115. The head 115 may be coupled to the tube 105, the shaft 110, or both. In general, components of the apparatus 100 may be coupled directly or indirectly. Components may also be coupled by virtue of physical proximity, being integral to a single structure, or being formed from the same piece of material. The components of the apparatus 100 may be formed from a variety of materials suitable for surgery, such as stainless steel. In some examples, some components may be constructed from sheet metal.

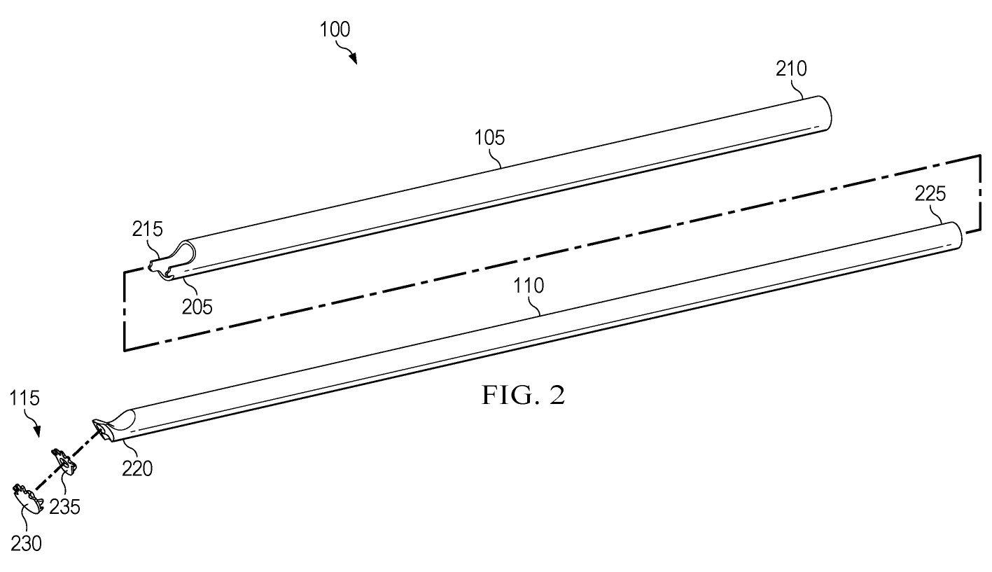

FIG. 2 is an assembly view of the apparatus 100 of FIG. 1, illustrating additional details that may be associated with some embodiments. For example, the tube 105 generally has a first end 205 and a second end 210. The tube 105 may have an opening 215 in the side near the first end 205, as illustrated in the example of FIG. 2. The shaft 110 of FIG. 2 generally has a first end 220 and a second end 225. The head 115 of FIG. 2 generally comprises a base 230 and a blade 235. In some embodiments, the base 230 may be coupled to the first end 205 of the tube 105, adjacent to the opening 215, and the blade 235 may be coupled to the first end 220 of the shaft 110. The opening 215 is generally configured to facilitate visibility of the head 115, and more particularly, the blade 235.

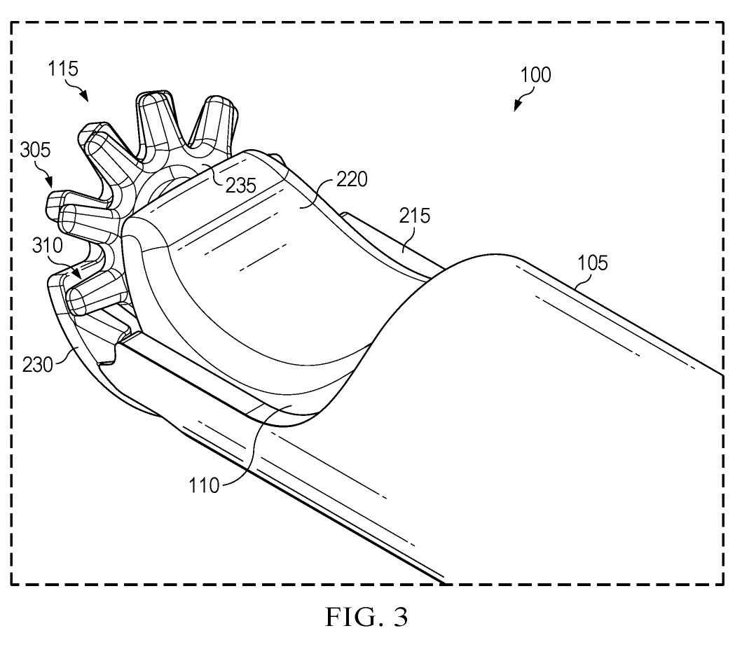

FIG. 3 is a detail view of the apparatus 100 of FIG. 1, illustrating additional details that may be associated with some embodiments. For example, the blade 235 may be disposed adjacent to, or at least partially in, the opening 215. The first end 220 of the shaft 110 of FIG. 3 has a concave profile and is generally aligned with the opening 215. The blade 235 can be coupled to the first end 220 so that the blade 235 is adjacent to or in the opening 215. The base 230 of FIG. 3 is coupled to the tube 105 so that the blade 235 is disposed between the base 230 and the first end 220. As illustrated in the example of FIG. 3, the base 230 may comprise a plurality of base teeth 305, and the blade 235 may comprise a plurality of blade teeth 310.

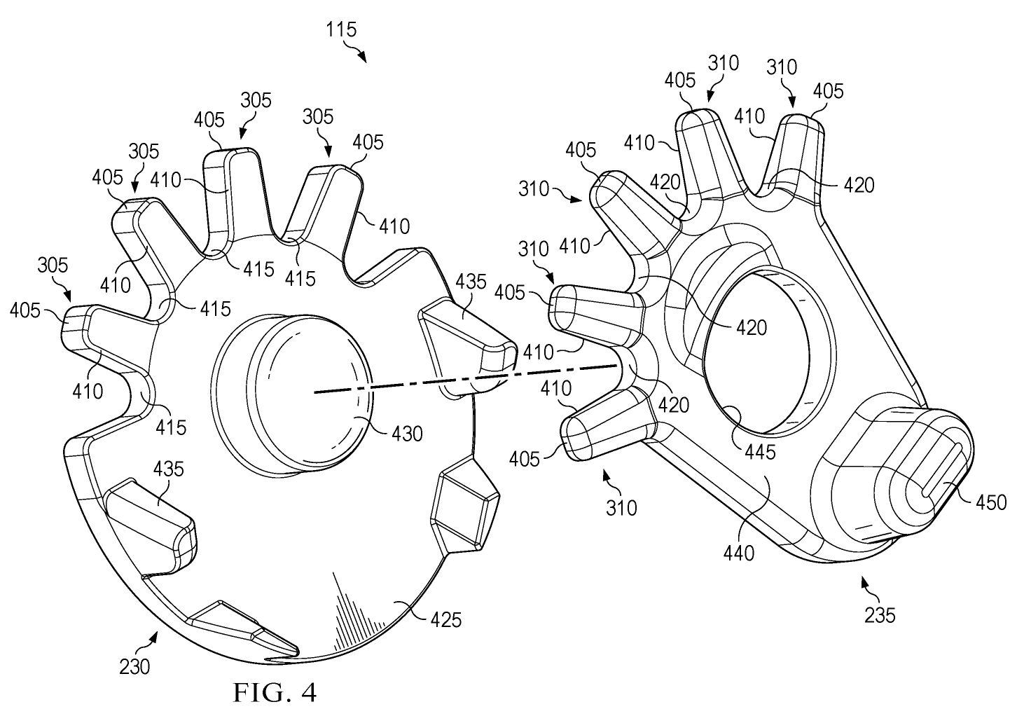

FIG. 4 is an assembly view of the head 115 of FIG. 3, illustrating additional details that may be associated with some embodiments. For example, each of the base teeth 305 extend from the base 230, and the blade teeth 310 extend from the blade 235. The base teeth 305 and the blade teeth 310 each generally has a leading edge 405 and at least one shearing edge 410. In general, the teeth may each have distinct shapes, profiles, and dimensions. In some examples, the teeth may be characterized as rounded, triangular, or some combination of both. The base teeth 305 may be separated by gaps 415, and the blade teeth 310 may be separated by gaps 420.

The base 230 of FIG. 4 also has a plate portion 425, a center post 430, and a pair of radial posts 435. In some examples, the base teeth 305 may be attached to the plate portion 425, and the center post 430 may be centrally disposed on the plate portion 425. In more particular examples, the base teeth 305 may be attached to the plate portion 425 in a radial configuration around the center post 430. The blade 235 of FIG. 4 generally comprises a plate portion 440, a hole 445 in the plate portion 440, and a blade post 450. In some examples, the blade teeth 310 may be attached to the plate portion 440 and may be attached in a radial configuration around the hole 445. The blade post 450 can be configured to couple the blade 235 to the shaft 110. For example, the shaft 110 may have a hole or receptacle configured to receive the blade post 450.

The leading edge 405 of each of the base teeth 305 and the blade teeth 310 may also have distinct shapes, profiles, and dimensions. For example, the leading edge 405 may be characterized as straight, curved, or some combination of both. The leading edge 405 of each of the base teeth 305 of FIG. 4 may be characterized, at least in part, as the edge, side, or surface having the greatest radial distance from the center post 430. The leading edge 405 of each of the blade teeth 310 of FIG. 4 may be characterized, at least in part, as the edge, side, or surface having the greatest radial distance from the hole 445. The shearing edge 410 may be characterized as transverse to the leading edge 405. The shearing edges 410 of the base teeth 305 may collectively define a base plane, and the shearing edges 410 of the blade teeth 310 may collectively define a blade plane.

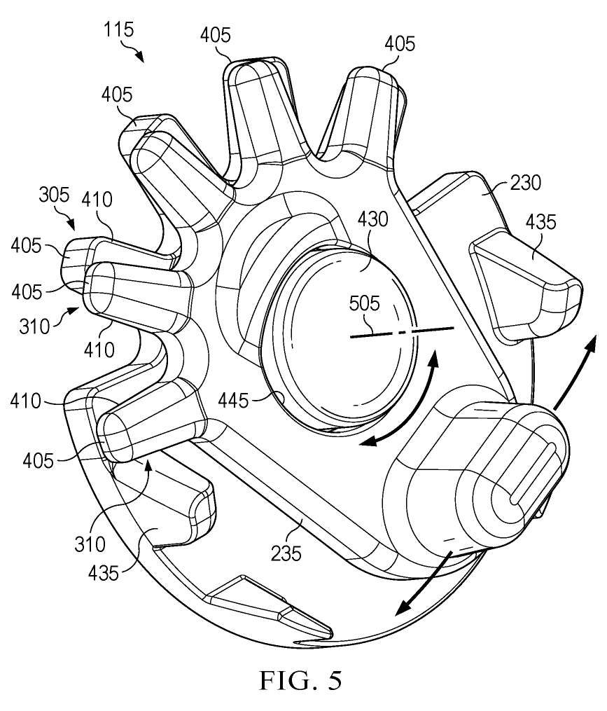

FIG. 5 is an isometric view of the head 115 of FIG. 4 in an assembled configuration, illustrating additional details that may be associated with some embodiments. For example, the hole 445 of the blade 235 of FIG. 5 is rotatably mounted on the center post 430 of the base 230. As assembled in the example of FIG. 5, the blade 235 may move relative to the base 230. For example, the blade 235 of FIG. 5 may rotate about an axis of rotation 505 that generally coincides with an axis of the center post 430, the hole 445, or both. In some examples, the radial posts 435 may limit the movement of the blade 235. Each of the blade teeth 310 of FIG. 5 are disposed adjacent to one of the base teeth 305 and may move relative to the base teeth 305 if the blade 235 is rotated around the center post 430.

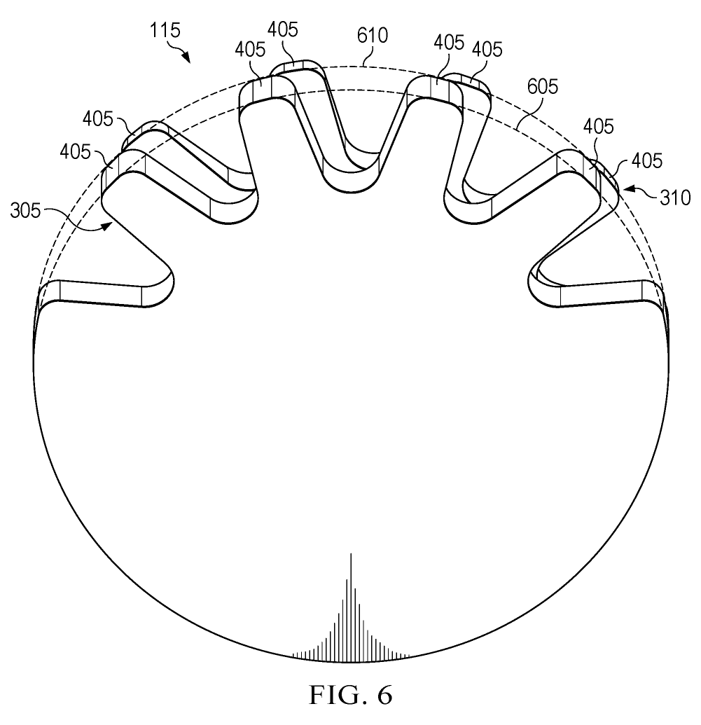

FIG. 6 is a bottom view of the head 115 of FIG. 5, illustrating additional details that may be associated with some embodiments. For example, the leading edge 405 of each of the base teeth 305 of FIG. 6 may collectively define a base profile 605. In FIG. 6, for example, the base profile 605 may be a curve that is tangential to the leading edge 405 of each of the base teeth 305. The base profile 605 may have a center of curvature that is coincident with the axis of rotation 505. In some embodiments, the curve may be characterized as an arc, as illustrated in FIG. 6. In some embodiments, the arc may be a circular arc, and in some examples, the arc may be characterized as a semicircle. In some embodiments, the outer curvature of the arc may approximate an outer peripheral edge curvature of the retina. Other curvatures are also contemplated.

The leading edge 405 of each of the blade teeth 310 may also define a blade profile 610, which may be similar to or substantially the same as the base profile 605. For example, the base profile 605 and the blade profile 610 of FIG. 6 define substantially congruent arcs.

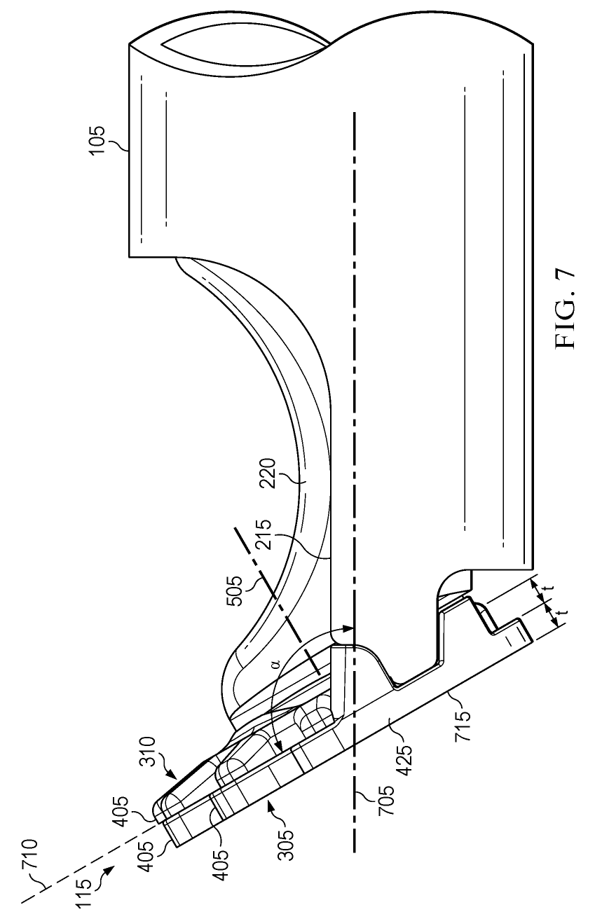

FIG. 7 is a side view of a portion of the apparatus 100 of FIG. 1, illustrating additional details that may be associated with some embodiments. For example, the tube 105 and the shaft 110 generally have a common longitudinal axis 705, and the interface between the base teeth 305 and the blade teeth 310 may define a shearing plane 710. In general, the shearing plane 710 may lie between and parallel to the base plane and the blade plane. In some examples, the shearing plane 710 lies between the shearing edges 410 of the base teeth 305 and the blade teeth 310. The shearing plane 710 may be orthogonal to the axis of rotation 505 and may be oriented at an angle α relative to the longitudinal axis 705. The angle α may generally vary according to surgical requirements in a range of about 90 to 160 degrees, for example in a range of approximately 90 to 130 degrees, for example, in a range of approximately 90 to 110 degrees. For example, the angle α may be approximately 110 degrees. Different angles may be used, for example, based on the geometry of the eye to be operated on. As also illustrated in the example of FIG. 7, the leading edge 405 may have a thickness t based the surgical need, condition of the membrane / retina, etc. For example, in some embodiments, the thickness t may be approximately in a range of 30 to 400 micrometers. In some embodiments, the thickness may be less than 400 micrometers. As a specific example, the thickness t may be approximately 200 micrometers. In other embodiments, the thickness t may be approximately 100 micrometers. Other dimensions are also contemplated (e.g., the thickness t may be in a range of approximately 30 to 100 micrometers, approximately in a range of 100 to 300 micrometers, approximately in a range of 300 to 400 micrometers, approximately in a range of 400 to 800 micrometers, approximately in a range of 800 to 1600 micrometers, etc.) The plate portion 425 and the base teeth 305 of FIG. 7 generally form a base surface 715. As shown in the example of FIG. 7, the base surface 715 may be generally parallel to the shearing plane 710. In some examples, it may be advantageous for the base surface 715 to be flat and smooth. In some examples, the base surface 715 may also have rounded edges.

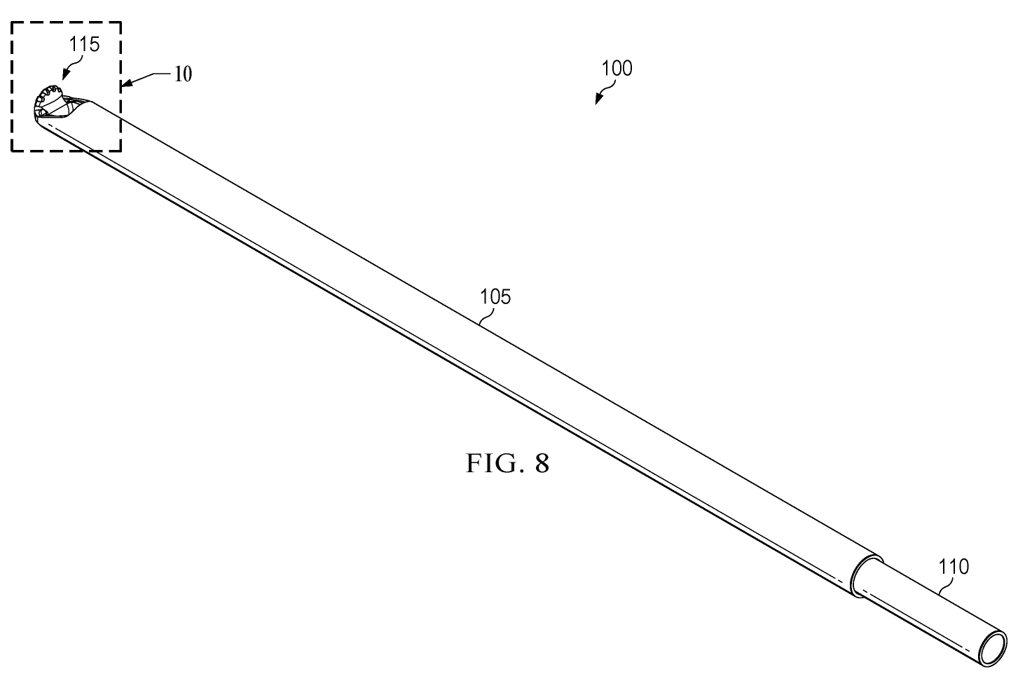

FIG. 8 is an isometric view of another example of the apparatus 100. The apparatus 100 of FIG. 8 may be similar to the apparatus 100 of FIG. 1 in many respects. For example, the apparatus 100 of FIG. 8 generally includes the tube 105, the shaft 110 rotatably disposed at least partially within the tube 105, and the head 115. The head 115 may be coupled to the tube 105, the shaft 110, or both.

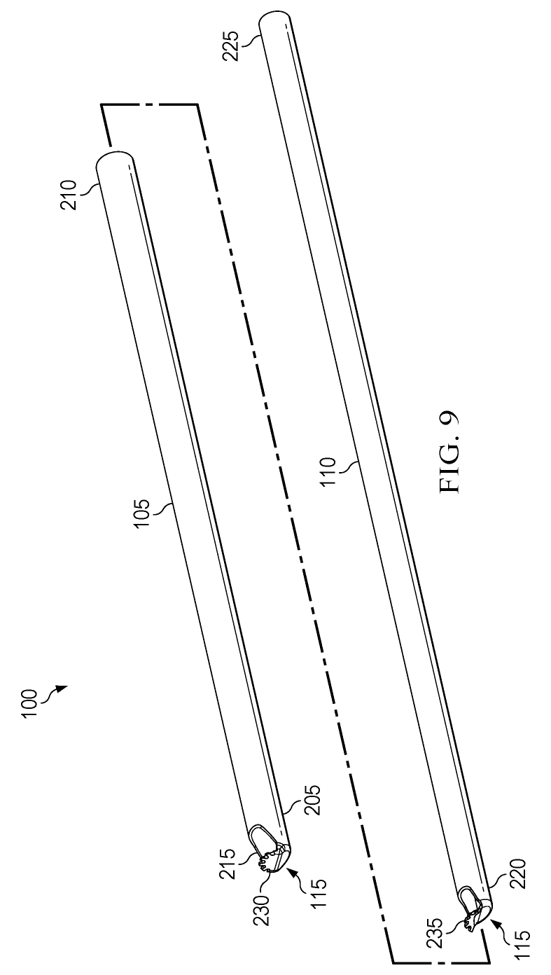

FIG. 9 is an assembly view of the apparatus 100 of FIG. 8, illustrating additional details that may be associated with some embodiments. For example, the base 230 of FIG. 9 may be coupled to the first end 205 of the tube 105, adjacent to the opening 215, and the blade 235 may be coupled to the first end 220 of the shaft 110.

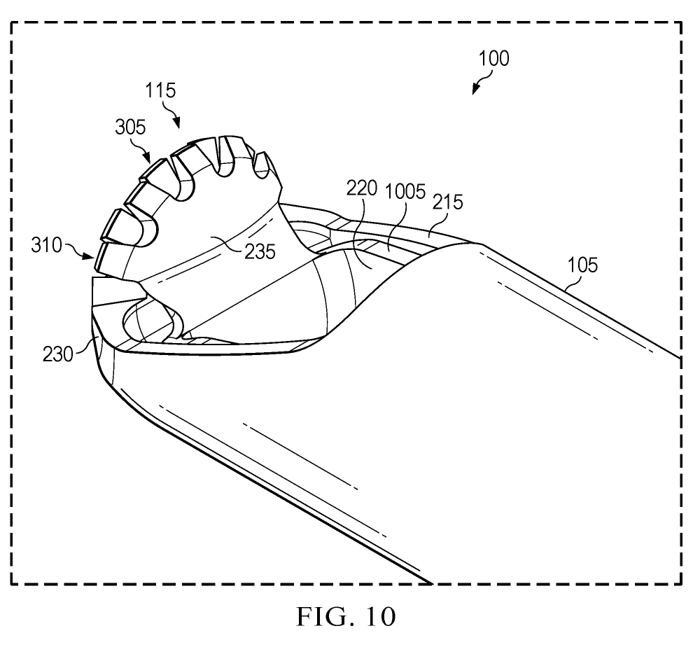

FIG. 10 is a detail view of the apparatus 100 of FIG. 8, illustrating additional details that may be associated with some embodiments. For example, the blade 235 may be disposed adjacent to, or at least partially in, the opening 215. In the example of FIG. 10, the first end 220 is hollow and has an opening 1005. The opening 1005 of FIG. 10 is generally aligned with the opening 215 of the tube 105. The blade 235 can be coupled to the first end 220 so that the blade 235 is adjacent to or in the opening 215.

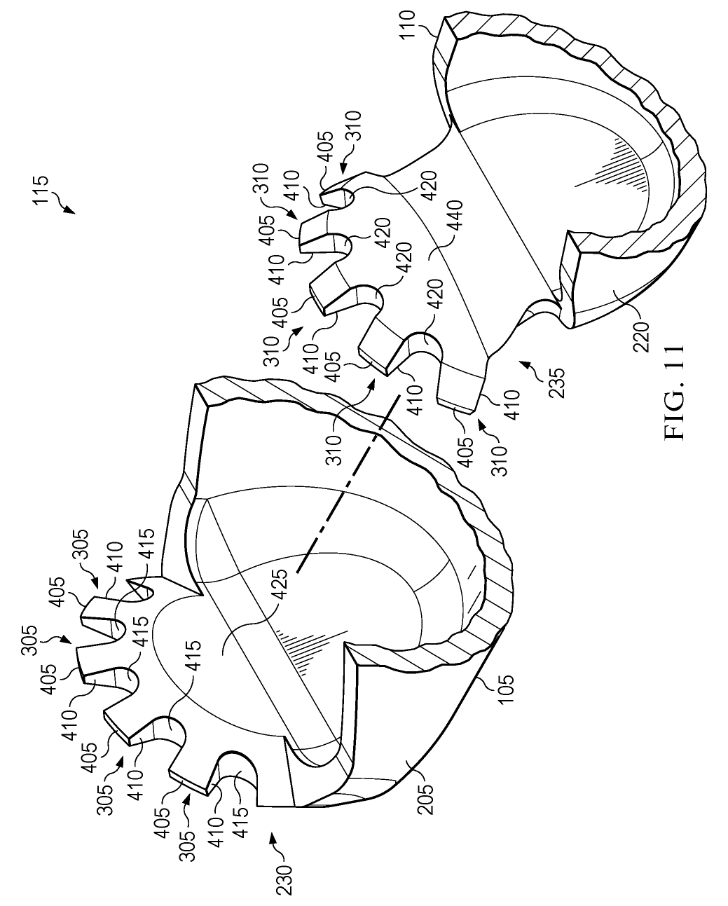

FIG. 11 is an assembly view of a portion of the apparatus 100 of FIG. 8, illustrating additional details that may be associated with some embodiments. As illustrated in the example of FIG. 11, some embodiments of the base 230 may be rigidly coupled to the first end 205 of the tube 105, and the blade 235 may be rigidly coupled to first end 220 of the shaft 110. For example, the plate portion 425 of the base 230 of FIG. 11 is rigidly coupled to the first end 205, and the plate portion 440 of the blade 235 of FIG. 11 is rigidly coupled to the first end 220. Each of the base teeth 305 can extend from the base 230, and the blade teeth 310 can extend from the blade 235. In general, the teeth may each have distinct shapes, profiles, and dimensions. In some examples, the teeth may be characterized as rounded, triangular, or some combination of both. The base teeth 305 may be separated by the gaps 415, and the blade teeth 310 may be separated by the gaps 420.

In some examples, the base teeth 305 may be attached to the plate portion 425. In more particular examples, the base teeth 305 may be attached to the plate portion 425 in a radial configuration around the plate portion 425. In some examples, the blade teeth 310 may be attached to the plate portion 440 and may be attached in a radial configuration around the plate portion 440.

The leading edge 405 of each of the base teeth 305 and the blade teeth 310 may also have distinct shapes, profiles, and dimensions. For example, the leading edge 405 may be characterized as straight, curved, or some combination of both. The leading edge 405 of each of the base teeth 305 of FIG. 11 may be characterized, at least in part, as the edge, side, or surface having the greatest radial distance from plate portion 425. The leading edge 405 of each of the blade teeth 310 of FIG. 11 may be characterized, at least in part, as the edge, side, or surface having the greatest radial distance from the plate portion 440.

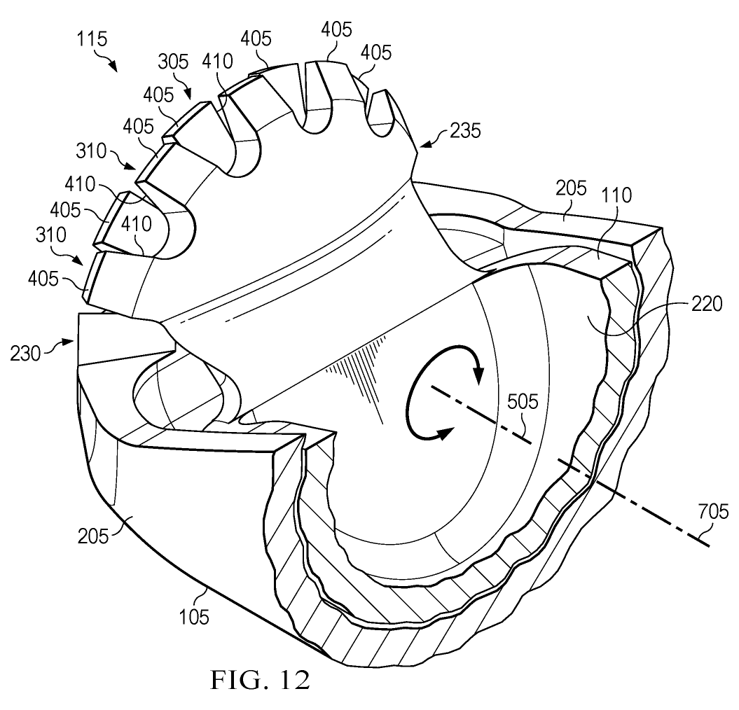

FIG. 12 is an isometric view of a portion of the apparatus of FIG. 8 in an assembled configuration, illustrating additional details that may be associated with some embodiments. For example, the first end 220 of the shaft 110 of FIG. 12 is rotatably disposed within the first end 205 of the tube 105, and the blade 235 extends from the first end 220 through the opening 215 of the tube 105. As assembled in the example of FIG. 12, the blade 235 may move relative to the base 230. For example, the shaft 110 can be rotated within the tube 105 about the axis of rotation 505, which generally coincides with the longitudinal axis 705. Rotating the shaft 110 can cause the blade 235 to also rotate about the axis of rotation 505 relative to the base 230. In some embodiments, the first end 205 of the tube 105 may limit the movement of blade 235. Each of the blade teeth 310 of FIG. 12 are disposed adjacent to one of the base teeth 305 and may move relative to the base teeth 305 if the blade 235 is rotated.

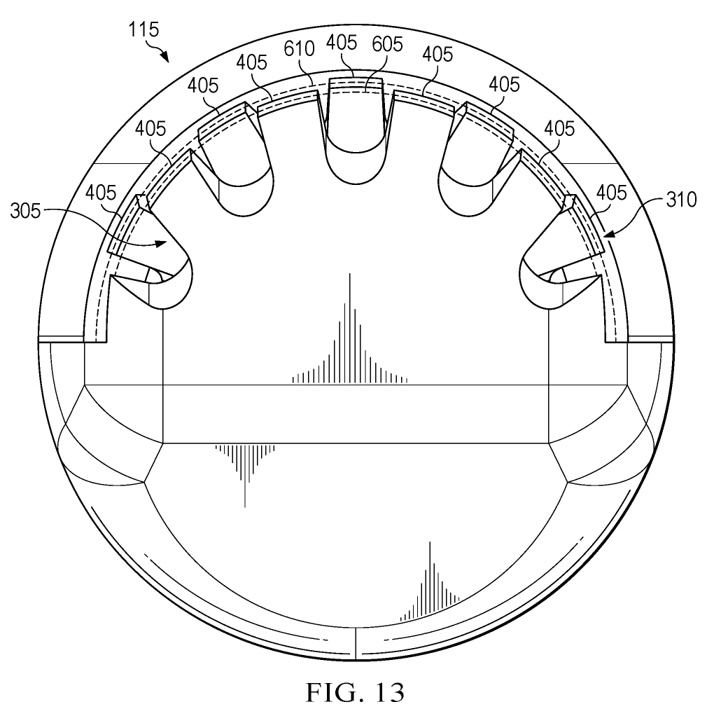

FIG. 13 is a bottom view of the head 115 of FIG. 12, illustrating additional details that may be associated with some embodiments. For example, the leading edge 405 of each of the base teeth 305 of FIG. 13 collectively define the base profile 605. As illustrated in FIG. 13, some embodiments of the base profile 605 may be a curve that is tangential to the leading edge 405 of each of the base teeth 305. In some embodiments, the curve may be characterized as an arc, as illustrated in FIG. 13. In some embodiments, the arc may be a circular arc, and in more particular examples, the arc may be characterized as a semicircle.

The leading edge 405 of each of the blade teeth 310 of FIG. 13 define the blade profile 610, which may be similar to or substantially the same as the base profile 605. For example, the base profile 605 and the blade profile 610 of FIG. 13 define substantially congruent arcs.

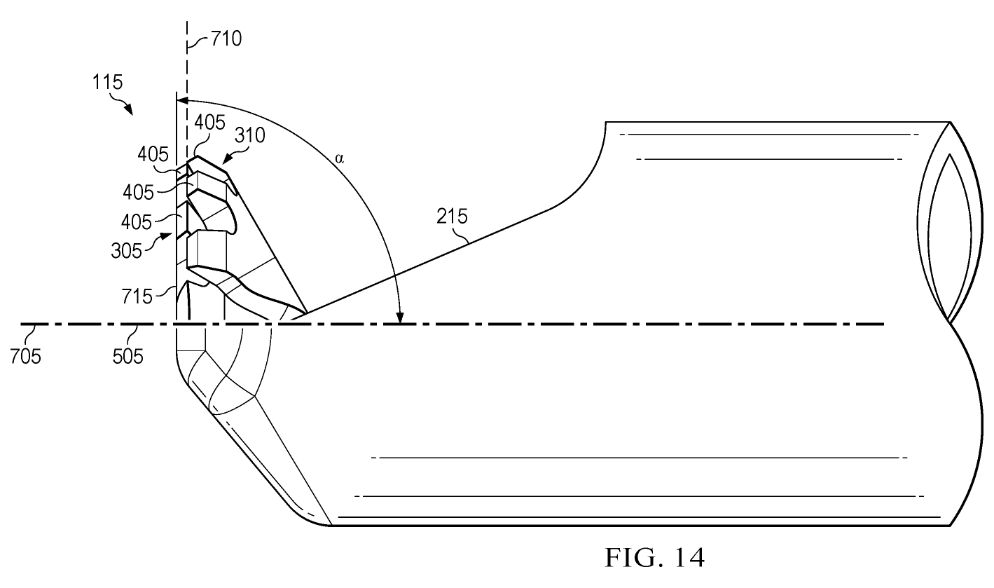

FIG. 14 is a side view of a portion of the apparatus 100 of FIG. 8, illustrating additional details that may be associated with some embodiments. For example, the interface between the base teeth 305 and the blade teeth 310 of FIG. 14 define the shearing plane 710. The shearing plane 710 may be orthogonal to the axis of rotation 505. In the example of FIG. 14, the shearing plane 710 is also orthogonal to the longitudinal axis 705.

In operation, the head 115 may be inserted in an eye after making an incision in the sclera and pars plana. In more particular examples, the head 115 may be inserted through an insertion cannula, which may be inserted through the incision. The head 115 may be placed against a retina, adjacent to membrane to be removed. The base surface 715 is generally configured to interface with the retina, and the opening 215 can provide visualization and access to the membrane. For example, the base surface 715 may be smooth and may have rounded edges to minimize risk of damage to the retina if the base surface 715 is placed in contact with the retina. In some examples, fixation of the base 230 relative to the blade 235 can also reduce risk of damage to the retina.

The membrane can be delaminated by cutting the connections between the retina and the membrane. For example, the blade 235 is generally configured to move relative to the base 230. In some embodiments, the blade 235 may be coupled to the shaft 110, substantially as described above, and the shaft 110 / blade 235 may be may be oscillated through manual or hand actuated motion (e.g., the user squeezing, pressing, or in some way actuating an actuator on the device to move the blade teeth). In some embodiments, the shaft may be coupled to a driver, such as an electromechanical motor configured to oscillate the shaft 110. The frequency of shaft rotation may be slow (e.g., in a range of 1 to 500 Hertz (Hz)) or may be fast (e.g., in a range of 1000 to 20,000 Hz or more) depending on the surgical need, condition of the membrane / retina, etc. Other frequencies are also contemplated (e.g., between 500 and 1000 Hz). In some embodiments, the frequency may be fixed or may be adjusted by the user. Oscillating the shaft 110, in turn, can cause the blade 235 to oscillate around the axis of rotation 505. In some examples, the blade teeth 310 can be oscillated parallel to the shearing plane. If inserted between the membrane and the retina, the movement of the blade teeth 310 relative to the base teeth 305 can shear connective tissue in the shearing plane 710. High oscillation frequency of the blade 235 and localized shearing action of the blade teeth 310 can minimize induced traction on the retina while shearing connective tissue. In some examples, the shaft 110 may be configured to aspirate severed tissue. For example, some embodiments of the shaft 110 may have an opening adjacent to the blade 235 that is configured to allow severed tissue to be removed.

Additionally, or alternatively, some examples of the head 115 can be used as a spatula to delaminate a membrane from a retina, even if the blade 235 is not oscillating. For example, the thickness t of the leading edges 405 may be configured to allow the head to be inserted between a membrane and a retina. In some examples, some or all the leading edges 405 may be sufficiently sharp to allow them to cut tissue between a membrane and a retina.

The systems, apparatuses, and methods described herein may provide significant advantages for delaminating a membrane to release traction on a retina. For example, the displacement of the blade 235 can be very small so that the shearing action only affects a small area, which can be very advantageous for reducing traction that can be caused by removal. The shape of the base teeth 305 and the blade teeth 310 can also reduce traction and increase efficiency.

Additionally, or alternatively, some embodiments of the head 115 can also be used for delamination without shearing, which can eliminate the need for an additional delamination tool. For example, some embodiments of the apparatus 100 can eliminate the need for using a vitrectomy probe, scissors, a spatula, and forceps for delamination. Use of such tools can affect a much larger area, which can also increase the traction force induced on a retina during delamination and lead to retinal breaks.

While shown in a few illustrative embodiments, a person having ordinary skill in the art will recognize that the systems, apparatuses, and methods described herein are susceptible to various changes and modifications that fall within the scope of the appended claims.

Moreover, descriptions of various alternatives using terms such as “or” do not require mutual exclusivity unless clearly required by the context, and the indefinite articles “a” or “an” do not limit the subject to a single instance unless clearly required by the context. Components may also be combined or eliminated in various configurations for purposes of sale, manufacture, assembly, or use.

The claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims.

What is claimed is:

1. An apparatus for eye surgery, the apparatus comprising:
a tube having an opening;
a base coupled to the tube adjacent to the opening, the base comprising a surface configured to interface with a retina;
a plurality of base teeth coupled to the base, wherein each of the base teeth comprises a leading edge, and the leading edges define a base profile that is a curve; and
a blade disposed at least partially within the opening between the tube and the base, wherein the blade is configured to move around an axis of rotation relative to the base teeth.

2. The apparatus of claim 1, wherein the base profile has a center of curvature that is coincident with the axis of rotation.

3. The apparatus of claim 2, wherein the base profile is an arc.

4. The apparatus of claim 2, wherein the base profile is a circular arc.

5. The apparatus of claim 2, wherein the base profile is a semicircle.

6. The apparatus of claim 1, further comprising a shaft coupled to the blade, the shaft rotatably disposed at least partially within the tube.

7. The apparatus of claim 1, wherein:
the base profile is an arc;
the blade comprises a plurality of blade teeth; and
the blade teeth are configured to oscillate relative to the base teeth.

8. The apparatus of claim 1, wherein:
the base profile is a first arc;
the blade comprises a plurality of blade teeth, each of the blade teeth comprises a leading edge, and the leading edges of the blade teeth define a blade profile;
the blade teeth are configured to oscillate relative to the base teeth; and
the blade profile is a second arc.

9. The apparatus of claim 1, wherein:
the base profile is a first arc;
the blade comprises a plurality of blade teeth, each of the blade teeth comprises a leading edge, and the leading edges of the blade teeth define a blade profile;
the blade teeth are configured to oscillate relative to the base teeth;
the blade profile is a second arc; and
the first arc and the second arc are congruent.

10. The apparatus of claim 1, wherein:
the base profile is a first arc;
the blade comprises a plurality of blade teeth, each of the blade teeth comprises a leading edge, and the leading edges of the blade teeth define a blade profile;
the blade teeth are configured to oscillate relative to the base teeth;
the blade profile is a second arc;
the first arc and the second arc are congruent.

11. The apparatus of claim 1, wherein:
the tube has a longitudinal axis;
the blade comprises a plurality of blade teeth;
the base teeth and the blade teeth define a shearing plane; and
the shearing plane is oriented at an obtuse angle relative to the longitudinal axis.

12. The apparatus of claim 11, wherein the angle is greater than 90 degrees.

13. An apparatus for eye surgery, the apparatus comprising:
a tube having an opening and a longitudinal axis;
a base coupled to the tube adjacent to the opening, the base comprising a surface configured to interface with a retina;
a plurality of base teeth coupled to the base;
a blade disposed at least partially within the opening between the tube and the base; and
a plurality of blade teeth coupled to the base;
wherein the base teeth and the blade teeth define a shearing plane, the shearing plane is oriented at an angle relative to the longitudinal axis that is greater than 90 degrees, and the blade teeth are configured to move around an axis of rotation relative to the base teeth and in the shearing plane.

14. The apparatus of claim 13, wherein:
each of the base teeth comprises a leading edge, and the leading edges of the base teeth define a first arc; and
each of the blade teeth comprises a leading edge, and the leading edges of the blade teeth define a second arc.

15. A system for eye surgery, the system comprising:
a tube having an opening and a longitudinal axis;
a shaft at least partially disposed within the tube;
a base coupled to the tube adjacent to the opening;
a plurality of base teeth coupled to the base;
a blade coupled to the shaft and disposed at least partially within the opening between the tube and the base;
a plurality of blade teeth coupled to the base, each of the blade teeth comprising a leading edge; and
a driver configured to be coupled to the shaft, the driver operable to rotate the shaft to oscillate the blade teeth parallel to a shearing plane defined between the blade teeth and the base teeth;
wherein each of the base teeth and the blade teeth comprise a leading edge, the leading edges of the base teeth define a first arc, the leading edges of the blade teeth define a second arc, and the shearing plane is oriented at an angle relative to the longitudinal axis that is greater than 90 degrees.
